(19) United States
(12) Patent Application Publication
(10) Pub. No.: US 2025/0152088 A1
(43) Pub. Date: May 15, 2025

(54) SYSTEM AND METHOD FOR MONITORING HUMAN SKIN

(71) Applicant: MVM ENTERPRISES GMBH, Zürich (CH)

(72) Inventors: Florian HERZOG, Zürich (CH); Rudolf Antun RUPEC, Zürich (CH)

(21) Appl. No.: 18/838,213

(22) PCT Filed: Feb. 1, 2023
(86) PCT No.: PCT/EP2023/052480

§ 371 Date: Aug. 13, 2024

(30) Foreign Application Priority Data
Feb. 14, 2022 (EP) 22156616.9

(51) Int. Cl. A61B 5/00; A61B 5/103; G06T 7/00; G06T 7/55; G06T 7/73; G06T 19/20; G06V 10/764; G06V 10/82; G06V 10/94; G06V 20/64; G16H 50/20

(52) U.S. Cl. CPC A61B 5/444; A61B 5/0077; A61B 5/1032; A61B 5/742; A61B 5/7475; G06T 7/0014; G06T 7/55; G06T 7/74; G06T 19/20; G06V 10/82; G16H 50/20; G06T 2200/24; G06T 2207/10024; G06T 2207/10028; G06T 2207/20081; G06T 2207/20084; G06T 2207/30088; G06T 2207/30201; G06T 2210/41; G06T 2219/2016; G06V 10/764; G06V 10/95; G06V 20/64; G06V 2201/03

(57) ABSTRACT

For monitoring human skin, a camera and a depth sensor record (SI) a plurality of 3D-surface images of a skin surface. Each 3D-surface image is taken from a different angle and a different distance with respect to the skin surface and comprises a plurality of pixels with depth information and RGB-color information. A computer system (1) detects (S2) skin surface features in the 3D-surface images and determines (S3) respective angular orientation and distance of the 3D-surface images, using the skin surface features and the depth SQ information. The computer system (1) generates (S4) a 6D-model of the skin surface, using the 3D-surface images and their respective angular orientation and distance. The 6D-model of the skin surface comprises a plurality of surface data points with 3D-coordinates and RGB-color information. The computer system (1) generates (S5) a dermatological evaluation of the 6D-model and renders (S6) the dermatological evaluation on a user interface.

### FIELD OF THE DISCLOSURE

The present disclosure relates to a system and a method for monitoring human skin. Specifically, the present disclosure relates to a computer system and computer-implemented method for monitoring human skin.

### BACKGROUND OF THE DISCLOSURE

Telemedicine has become more common not least because of the widespread availability and accessibility of the Internet. Even for medical consultation and initial diagnoses, telemedicine has become more acceptable and, particularly during the Corona pandemic or for patients in remote location, almost a necessity. This is also the case in dermatology where online websites are available to receive from patients photographs of their skin for a diagnosis and recommendation of treatment. For patients it seems quite simple to record and upload such photographs using their mobile phones and/or personal computers. This kind of telemedicine appears to be very practical, because it makes it much easier for patients to reach out and get care for a skin condition or symptom without leaving their home. Nevertheless, while a remote dermatological diagnosis based on photographs may be suitable and correct in some cases, it is not considered accurate because of the limited reliability and informa-tional content of photographs.

### SUMMARY OF THE DISCLOSURE

It is an object of this disclosure to provide a system and a method for monitoring human skin. In particular, it is an object of the present disclosure to provide a computer-implemented method and a computer system for monitoring human skin system, which method and system do not have at least some of the disadvantages of the prior art.

According to the present disclosure, these objects are addressed by the features of the in-dependent claims. In addition, further advantageous embodiments follow from the dependent claims and the description.

According to the present disclosure, the above-mentioned objects are particularly achieved in that a computer-implemented method of monitoring human skin comprises a camera and a depth sensor recording a plurality of 3D-surface images of a skin surface. Each 3D-surface image is taken from a different angle and a different distance, with respect to the skin surface, and comprises a plurality of pixels with depth information and RGB-color information (3D=three-dimensional; RGB=Red, Green, Blue). A computer system detects skin surface features in the 3D-surface images. Using the skin surface features and the depth information, the computer system determines respective angular orientation and distance of the 3D-sur-face images. Using the 3D-surface images and their respective angular orientation and distance, the computer system generates a 6D-model of the skin surface (6D=six-dimensional).

The 6D-model of the skin surface comprises a plurality of surface data points. Each surface data point comprises 3D-coordinates and RGB-color information. The computer system generates a dermatological evaluation of the 6D-model of the skin surface and renders the dermatological evaluation on a user interface. For example, the plurality of 3D-surface images of the skin surface is recorded using a camera and a depth sensor of a mobile electronic device, for example a hand-held electronic device, e.g. a mobile communication device, such as mobile phone (a smart phone or a smart watch) or a tablet computer.

In an embodiment, the computer system determines from the 3D-surface images a body part and uses an initial coordinate system and respective feature outlines, associated with the body part, for detecting the skin surface features in the 3D-surface images.

In an embodiment, the computer system selects from the plurality of 3D-surface images a 3D-reference image. The 3D-reference image has a coordinate system with an angular orientation comparatively closest to the initial coordinate system. The computer system uses the 3D-reference image to generate the 6D-model of the skin surface.

In an embodiment, the computer system generates the 6D-model of the skin surface by determining adjacent 3D-surface images, starting with the 3D-reference image, whereby two adjacent 3D-surface images have a comparatively closest angular orientation and distance to each other, and rotating and translating adjacent 3D-reference images towards the 3D-reference image, starting with the 3D-surface image adjacent to the 3D-reference image, to match their respective angular orientation and distance.

In an embodiment, the computer system includes in the dermatological evaluation a probabilistic ranking of dermatological diagnoses related to skin diseases, skin issues, and/or skin types. For example, the dermatological evaluation comprises a list of skin diseases, skin issues, and/or skin types, and a probabilistic ranking of these skin diseases, skin issues, and/or skin types.

In an embodiment, the computer system generates the dermatological evaluation using the 6D-model of the skin surface as input to a neural network. The neural network is trained to identify skin diseases, skin issues, and/or skin types, using a plurality of 6D-models of skin surfaces from a plurality of people. The 6D-models of skin surfaces comprise a plurality of surface data points with 3D-coordinates and RGB-color information.

In an embodiment, the computer system generates a 5D-map of the skin surface, by applying a projection to the 6D-model of the skin surface (5D=five-dimensional). The 5D-map of the skin surface comprises a plurality of map data points. Each map data point comprises 2D-coordinates and RGB-color information (2D=two-dimensional). The computer system generates the dermatological evaluation using the 5D-map of the skin surface as input to a neural network. The neural network is trained to identify skin diseases, skin issues, and/or skin types, using a plurality of 5D-maps of skin surfaces from a plurality of people. The 5D-maps of skin surfaces comprise a plurality of map data points with 2D-coordinates and RGB-color information.

In an embodiment, the computer system generates one or more 6D-sub-models of the skin surface, by extracting one or more areas of the 6D-model of the skin surface. Each of the 6D-sub-models of the skin surface comprises a plurality of surface data points of the respective area. Each surface data point comprises 3D-coordinates and RGB-color information. The computer system generates the dermatological evaluation using the one or more 6D-sub-models of the skin surface as input to a neural network. The neural network is trained to identify skin diseases, skin issues, and/or skin types, using a plurality of one or more of the 6D-sub-models of skin surfaces from a plurality of people. The one or more 6D-sub-models of skin surfaces comprise a plurality of surface data points of the respective area with 3D-coordinates and RGB-color information.

In an embodiment, the computer system generates one or more 6D-sub-models of the skin surface and/or a 5D-map of the skin surface. The computer system generates the one or more 6D-sub-models of the skin surface by extracting one or more areas of the 6D-model of the skin surface. Each of the 6D-sub-models of the skin surface comprises a plurality of sur-face data points of the respective area. Each surface data point comprises 3D-coordinates and RGB-color information. The computer system generates the 5D-map of the skin surface by applying a projection to the 6D-model of the skin surface. The 5D-map of the skin surface comprises a plurality of map data points. Each map data point comprises 2D-coordinates and RGB-color information. The computer system generates the dermatological evaluation using the 6D-model of the skin surface, the one or more 6D-sub-models of the skin surface, and/or the 5D-map of the skin surface, as input to a neural network. The neural network is trained to identify skin diseases, skin issues, and/or skin types, using a plurality of 6D-models of skin surfaces from a plurality of people, a plurality of one or more 6D-sub-models of skin surfaces from a plurality of people, and/or a plurality of 5D-maps of skin surfaces from a plurality of people.

In an embodiment, the computer system uses the 6D-model of the skin surface to determine skin surface characteristics. The skin surface characteristics include wrinkles, wrinkle dimensions, pores, pore dimensions, milia, nodules, nodule dimensions, nodule shapes, macula, macula dimensions, macula shapes, papule, papule dimensions, papule shapes, plaque, plaque dimensions, plaque shapes, pustules, pustule dimensions, blisters, blister dimensions, wheal, wheal dimensions, wheal shapes, comedo, erosions, erosion dimensions, erosion shapes, ulcer, ulcer dimensions, ulcer shapes, crust, scale, scale types, rhagade, and/or atrophy. The computer system uses the skin surface characteristics to generate the dermatological evaluation.

In an embodiment, upon completion of a first dermatological evaluation for a first 6D-model of the skin surface, the computer system stores in a data storage the first 6D-model of the skin surface and the first dermatological evaluation. Upon receiving a second 6D-model of the skin surface, the computer system identifies in the data storage the first dermatological evaluation, by comparing the second 6D-model of the skin surface to the first 6D-model of the skin surface. Upon completion of a second dermatological evaluation for the second 6D-model of the skin surface, the computer system generates a tracking report, by comparing the second dermatological evaluation to the first dermatological evaluation, and renders the tracking report on the user interface.

In an embodiment, the computer system renders the 6D-model of the skin surface on a display, receives evaluation data from a user via a user interface, and generates the dermatological evaluation using the evaluation data.

In an embodiment, an electronic device of the computer system transmits the 6D-model of the skin surface via communication network to a processing system of the computer system. The processing system uses the 6D-model of the skin surface received from the electronic device to generate the dermatological evaluation. The processing system transmits the dermatological evaluation to the electronic device.

In addition to the method of monitoring human skin, the present disclosure also relates to a computer system. The computer system comprises a camera, a depth sensor, a user interface, and one or more processors. The one or more processors are configured to control the camera and the depth sensor to record a plurality of 3D-surface images of a skin surface. Each 3D-surface image is taken from a different angle and a different distance with respect to the skin surface and comprises a plurality of pixels with depth information and RGB-color information. The one or more processors are configured to detect skin surface features in the 3D-surface images. The one or more processors are configured to determine respective angular orientation and distance of the 3D-surface images, using the skin surface features and the depth information. The one or more processors are configured to generate a 6D-model of the skin surface, using the 3D-surface images and their respective angular orientation and distance. The 6D-model comprises a plurality of surface data points, each surface data point comprises 3D-coordinates and RGB-color information. The one or more processors are configured to generate a dermatological evaluation of the 6D-model of the skin sur-face and to render the dermatological evaluation on the user interface.

The one or more processors are configured to perform the above-described method of monitoring human skin.

In an embodiment, the computer system comprises an electronic device and a processing system. The electronic device includes one or more of the processors, which are configured to transmit the 6D-model of the skin surface via communication network to the processing system. The processing system includes one or more processors, which are configured to generate the dermatological evaluation, using the 6D-model of the skin surface received from the electronic device, and to transmit the dermatological evaluation to the electronic device. For example, the electronic device is a mobile electronic device, for example a hand-held electronic device, e.g. a mobile communication device, such as mobile phone (a smart phone or a smart watch) or a tablet computer. For example, the computerized processing system is a computerized server system, e.g. a cloud-based computer system.

In addition to the method and computer system for monitoring human skin, the present disclosure also relates to a computer program product comprising computer program code for controlling a processor of an electronic device. In particular, the present disclosure relates to a computer program product comprising a computer readable medium having stored thereon computer program code, e.g. a non-transitory computer readable medium. The computer program code is configured to control a processor of an electronic device comprising a camera, a depth sensor, and a user interface connected to the processor, such that the processor controls the camera and the depth sensor to record a plurality of 3D-surface images of a skin surface of a human skin. Each 3D-surface image is taken from a different angle and a different distance, with respect to the skin surface, and comprises a plurality of pixels with depth information and RGB-color information. The computer program code is further configured to control the processor of the electronic device, such that the processor detects skin surface features in the 3D-surface images; determines respective angular orientation and distance of the 3D-surface images, using the skin surface features and the depth information; generates a 6D-model of the skin surface, using the 3D-surface images and their respective angular orientation and distance, whereby the 6D-model of the skin surface comprises a plurality of surface data points, each surface data point comprising 3D-coordinates and RGB-color information; transmits the 6D-model of the skin surface to a computerized processing system; receives from the computerized processing system a dermatological evaluation of the 6D-model of the skin surface; and renders the dermatological evaluation on the user interface. For example, the electronic device is a mobile electronic device, for example a hand-held electronic device, e.g. a mobile communication device, such as mobile phone (a smart phone or a smart watch) or a tablet computer.

The computer program code is further configured to control the processor of the electronic device to perform the above-described method of monitoring human skin.

In addition to the method and computer system for monitoring human skin, the present disclosure also relates to a computer program product comprising computer program code for controlling one or more processors of a computerized processing system. In particular, the present disclosure relates to a computer program product comprising a computer readable medium having stored thereon computer program code, e.g. a non-transitory computer readable medium. The computer program code is configured to control the one or more processors of the computerized processing system to receive from an electronic device a 6D-model of a skin surface of a person, whereby the 6D-model of the skin surface comprises a plurality of surface data points, each surface data point comprising 3D-coordinates and RGB-color information. The computer program code is further configured to control the one or more processors of the computerized processing system to generate a dermatological evaluation of the 6D-model of the skin surface of the person, and to transmit the dermatological evaluation to the electronic device. For example, the computerized processing system is a computerized server system, e.g. a cloud-based computer system.

The computer program code is further configured to control the one or more processors of the computerized processing system to perform the above-described method of monitoring human skin.

### BRIEF DESCRIPTION OF THE DRAWINGS

The present disclosure will be explained in more detail, by way of example, with reference to the drawings in which:

FIG. 1 shows a block diagram illustrating schematically a computer system for monitoring human skin.

FIG. 2 shows a block diagram illustrating schematically a computer system for monitoring human skin, comprising an electronic device and a computerized processing system.

FIG. 3 shows a flow diagram illustrating an exemplary sequence of steps for monitoring human skin by a computer system.

FIG. 4 shows a flow diagram illustrating an exemplary sequence of steps for monitoring human skin by a computer system comprising an electronic device and a computerized processing system.

FIG. 5 shows a flow diagram illustrating an exemplary sequence of steps for generating a dermatological evaluation from a 6D-model of a skin surface.

FIG. 6 shows a flow diagram illustrating an exemplary sequence of steps for generating a tracking report for related dermatological evaluations of 6D-models of a skin surface.

FIG. 7 shows a schematic illustration of an initial coordinate system and feature outlines associated with a body part, particularly a human face, for detecting skin surface features.

FIG. 8 shows a schematic illustration of a body part, particularly a human face, with an overlaid initial coordinate system and feature outlines associated with the body part, particularly facial features, for detecting skin surface features.

FIG. 9 shows a tree-structured graph for labeling skin diseases and skin issues.

### DETAILED DESCRIPTION OF THE EMBODIMENTS

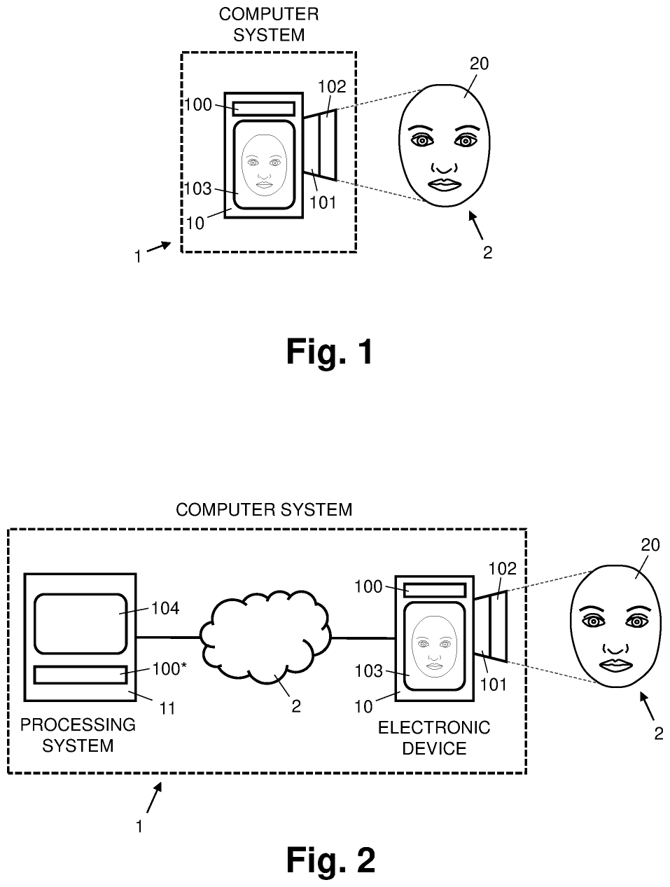

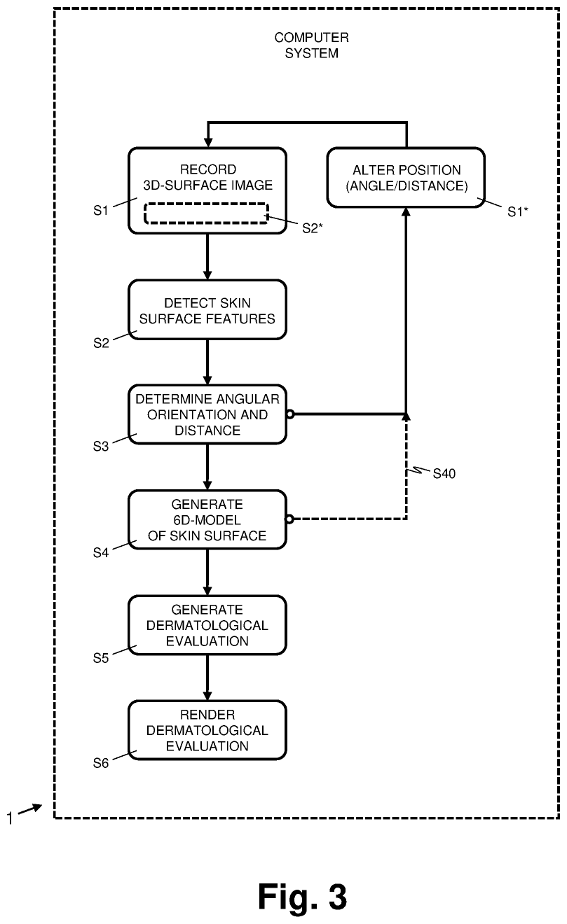

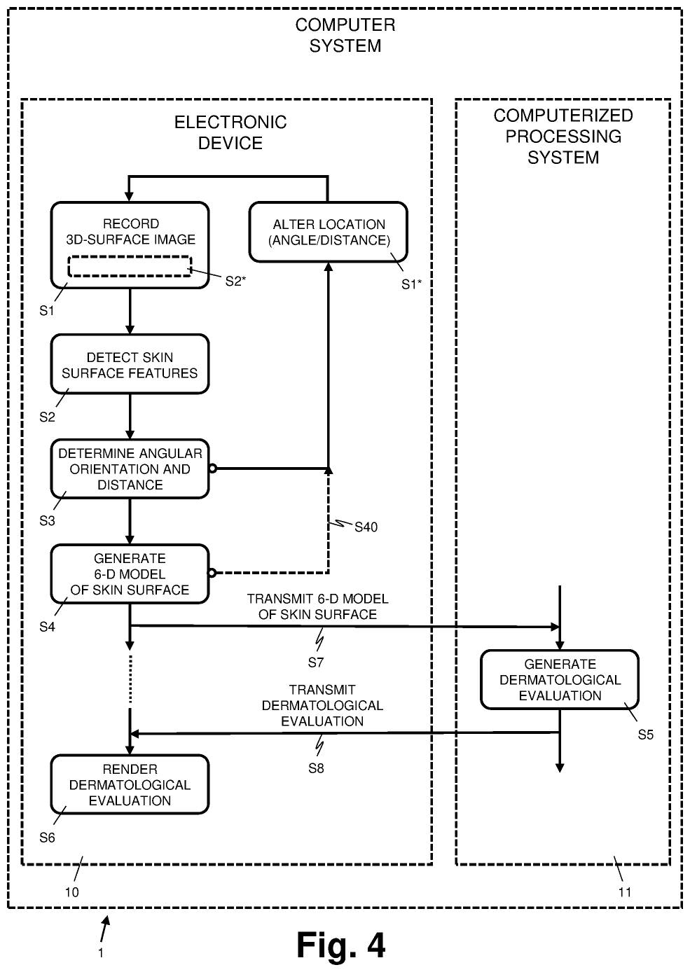

In FIGS. 1 to 4, reference numeral 1 refers to computer system. Depending on the embodiment and configuration, the computer system 1 comprises one or more processors 100, 100* arranged in one or more devices. FIGS. 1 and 3 illustrate embodiments where the one or more processors 100 of the computer system 1 are arranged in one electronic device 10. FIGS. 2 and 4 illustrate embodiments where the one or more processors 100, 100* of the computer system 1 are arranged in more than one device, particularly in the electronic device and in the computerized processing system 11.

The electronic device 10 is a personal computing device, such as a personal computer, e.g. a laptop or desktop computer, or a mobile computing device, e.g. a handheld device such as a mobile phone (smart phone or a smart watch) or a tablet computer. As illustrated schematically in FIGS. 1, 2, and 8, the computer system 1 comprises a digital camera 101, a depth sensor 102, and a user interface 103. In the embodiments illustrated in FIGS. 1, 2, and 8, the electronic device 10 is shown schematically to include the digital camera 101, the depth sensor 102, and the user interface 103. Nevertheless, the person skilled in the art will understand that components of the electronic device 10 can be arranged in separate housings, connected to the processor 100 of the electronic device 10 via wireless or wired communication link(s); particularly, the digital camera 101 and the depth sensor 102 can be arranged in a separate housing or unit, e.g. a handheld camera unit, and/or the user interface 103 can be arranged in separate units, such as a screen terminal and/or a keyboard, a computer mouse, a touchpad, and the like. The camera 101, the depth sensor 102, and the user interface 103 are connected to the processor 100. The user interface 103 comprises a display, a keyboard, a touch sensitive display, a microphone, and/or a speaker. The depth sensor 102 comprises an infrared, a LIDAR (Light Detection and Ranging), an ultrasound, or any other suitable depth or distance measuring sensor. Modern mobile phones have built-in combina-tions of digital camera and depth sensor. For example, the depth sensor 102 has a depth resolution of at least 1 mm to 0.1 mm or better. The processor 100 is configured to control the digital camera 101 and the depth sensor 102 to capture 3D-surface images with RGB-color (Red, Green, Blue) information, e.g. 8-bit to 16-bit color information per color. The 3D-surface images are defined in a local coordinate system, specifically in camera coordinates with respect to a camera coordinate system. The definition of the 3D-surface images in the local coordinate system includes camera specific parameters, such as focal lengths, focus, and device specific data such as angular rotation vis-A-vis the earth gravity, angular acceleration and speed as well as lateral acceleration and speed, etc. The pixel data together with the depth data and the camera specific parameters (e.g. focal length) is used to compute the 3D-surface image. The 3D-surface images have a 2D-resolution of Full-HD (High Definition) or more (amounting to at least 1920×1080=2073800 pixels)) and a down sample resolution of 640 by 480 pixels (640×480=307200 pixels). The depth or distance measurement has an accuracy or resolution of 1 mm to 0.1 mm or better, and a capturing resolution of at least 640 by 480 (=307200) depth measures. The person skilled in the art will understand, that lower resolutions of the digital camera 101 and/or the depth sensor 102 can be employed when the total number of 3D-surface images taken is increased to obtain a defined target resolution of a 6D-model of the skin surface, described and defined later in more detail. As illustrated schematically in FIGS. 1, 2 and 8, the digital camera 101 and the depth sensor 102 are configured to capture and record 3D-surface images with RGB-color information of the skin surface 20 of a human body part 2, in the example of FIGS. 1, 2 and 8, a facial skin surface of a human face 2. As illustrated in FIG. 8, the 3D-surface images are taken from different positions P1, P2 with different angles α1/β1, α2/β2 and different distances d1, d2 with respect to the skin surface 20, more specifically, with respect to a reference coordinate system C (x, y, z) and reference point R of the skin surface 20, as will be described later in more detail.

The computerized processing system 11 comprises one or more computers, e.g. computerized servers, with one or more processors 100*.

As illustrated in FIG. 2, the electronic device 10 and the computerized processing system 11 are connected via a communication network 2. The electronic device 10 and the computerized processing system 11 are configured for data communication via the communication network 2. The communication network comprises local area networks (LAN), wireless local area networks (WLAN), mobile radio networks, such as GSM (Global System for Mobile Communication) or UMTS (Universal Mobile Telephone System) networks, and/or the Internet.

In the following paragraphs, described with reference to FIGS. 3 to 8 are possible se-quences of steps performed by the computer system 1 or its one or more processors 100, 100* for monitoring human skin.

As illustrated in FIGS. 3 and 4, in step S1, the computer system 1 captures and records a 3D-surface image with RGB-color information of the skin surface 20 of a human body part 2.

More specifically, the computer system 1 or its processor 100, respectively, controls the digital camera 101 and the depth sensor 102 to capture and store the 3D-surface image with RGB-color information.

In step S1*, the respective position of the digital camera 101 and depth sensor 102 with regards to the skin surface 20 or body part 2 is altered. Either the position of the camera 101 and depth sensor 102 is changed or the body part 2 is moved. In an embodiment, the computer system 1 or its processor 100, respectively, generates and renders on a user interface 103 instructions on how to alter the respective position, e.g. visually on a display and/or acoustically via a speaker. Alternatively, the computer system 1 or its processor 100, respectively, controls an electric motor to change, e.g. rotate, the digital camera 101 and the depth sensor 102, to a different respective position.

The steps S1 and S1* are repeated to capture a plurality of 3D-surface image with RGB-color information of the skin surface 20 of a human body part 2 from different positions P1, P2 with different angles α1/β1, α2/β2 and different distances d1, d2 with respect to the skin surface 20. For example, the plurality of 3D-surface images are captured by a user moving the camera 101 and depth sensor 102 of a hand-held electronic device 10 in continuous movement with a distance of 10 to 30 centimeters over the area of the skin surface 20 to be monitored, e.g. in front of the face or a particular area of the face. The person skilled in the art will understand that for capturing larger skin areas, e.g. full body images, the distance can be increased, e.g. up to 150 cm. For example, a number of approximately 30 3D-surface images are taken per second during a recording period of approximately 5 to 25 seconds, amounting to approximately 150 to 750 captured and stored 3D-surface images. Nevertheless, the person skilled in the art will understand that lower or higher number of 3D-surface images, e.g. 1 to 60, can be taken during shorter or longer recording periods, e.g. 1 to 90 seconds, respectively.

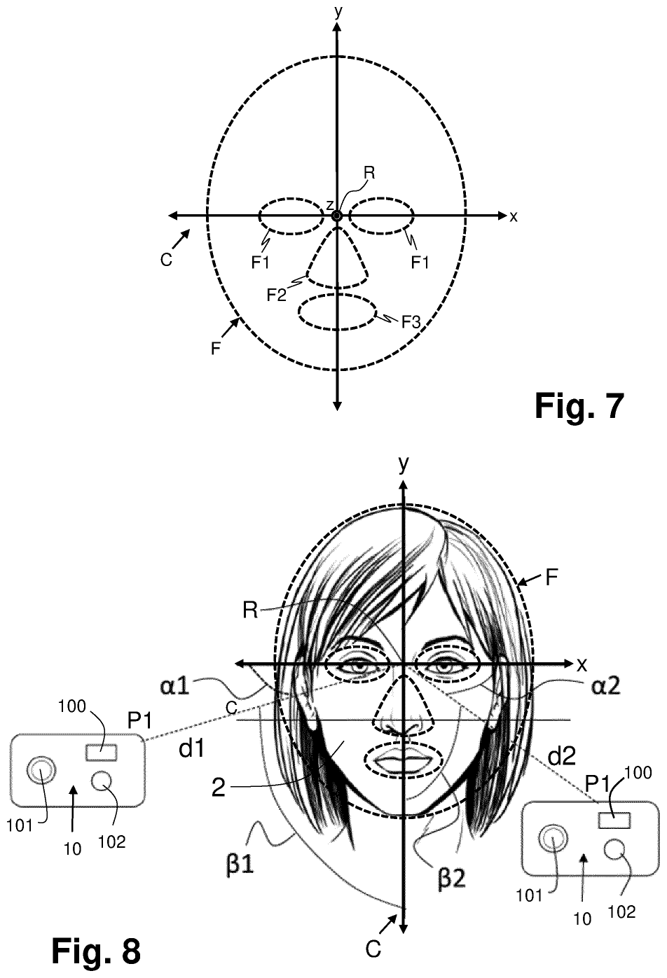

As illustrated schematically in FIGS. 3 and 4, in optional step S2*, the computer system 1 or its processor 100, respectively, determines the body part 2. In an embodiment the body part 2 is selected prior to step S1 by a user via the user interface 103. In another embodiment, the body part 2 is determined by the computer system 1 or its processor 100, respectively, from the first 3D-surface image(s), e.g. as part of step S1. The computer system 1 or its processor 100, respectively, determines an initial coordinate system C, e.g. a 3D xyz-coordinate system C with reference point R, and feature outlines F, F1, F2, F3 of skin surface features (“landmarks”) associated with the body part 2 and its skin surface 20, as illustrated schematically in FIGS. 7 and 8. The feature outlines F, F1, F2, F3 are three-dimensional (3D) and determine the location and approximate area or shape of characteristic features of the respective body part 2 in the initial coordinate system C. For example, for a human face, the feature outlines define the outline of the face F, the outline of the eyes F1, the outline of the nose F2, and the outline of the mouth F3, as illustrated in FIGS. 7 and 8. In the case of a human foot, the feature outlines define the outline of the toes and/or toenails. In the case of a human hand, the feature outlines define the outline of the fingers and/or fingernails. In a further embodiment, the computer system 1 or its processor 100, respectively, receives from the user via the user interface 103 commands and/or specifications of at least part of the initial coordinate system C. For example, if the back is the determined/selected body part 2, the computer system 1 or its processor 100, respectively, receives from the user via the user interface 103 user commands and/or specifications defining a selected area of the back to be captured.

In step S2, the computer system 1 or its processor 100, respectively, detects skin surface features (‘landmarks’) in the recorded 3D-surface images of the skin surface 20. More specifically, the computer system 1 or its processor 100, respectively, detects the skin surface features using the feature outlines F, F1, F2, F3 associated with the determined body part 2.

The skin surface features are defined in the local coordinate system, specifically in camera coordinates with respect to the camera coordinate system. In the case of the face, the computer system 1 or its processor 100, respectively, detects the outline of the face F, the outline of the eyes F1, the outline of the nose F2, and/or the outline of the mouth F3. In an embodiment, the computer system 1 or its processor 100, respectively, overlays the detected skin surface features onto the image captured by the digital camera 101 and shown on the display of the user interface 103, as depicted in FIG. 8.

In step S3, the computer system 1 or its processor 100, respectively, determines the angular orientation and the distances of the captured 3D-surface images of the skin surface 20. More specifically, the computer system 1 or its processor 100, respectively, determines the angular orientation and the distances of the captured 3D-surface images using the detected skin surface features and the depth information of the 3D-surface images. The angular orientation and the distance of a captured 3D-surface image is defined based on its local coordinate system with respect to a global coordinate system, more specifically the reference coordinate system C (x, y, z). For example, the angular orientation and the distance of a captured 3D-surface image are defined by way of respective angles α1/β1 (or α2/β2) and distance d1 (or d2) with respect to the reference coordinate system C (x, y, z) and reference point R of the skin surface 20, as illustrated schematically in FIG. 8. The computer system 1 or its processor 100, respectively, stores the captured 3D-surface images of the skin surface 20 and their respective angles α1/β1, α2/β2 and distances d1, d2 in local memory. Once the computer system 1 or its processor 100, respectively, determines that sufficient 3D-surface images have been captured for generating a 6D-model of the skin surface 20 of the body part 2, processing continues in step S4. For example, the number of captured 3D-surface images is sufficient, if a minimum resolution threshold is exceeded, e.g. a minimum number of 3D-surface images from a minimum number of different positions P1, P2, to ensure a sufficient coverage and resolution of the 6D-model of the skin surface 20 (see also further respective explanations in connection with step S5). Otherwise, processing continues in step S1* by altering the respective positioning of the digital camera 101 and the skin surface 20 of the body part 2 for capturing another 3D-surface image in step S1 from another position P1, P2. For example, the target resolution for the 6D-model of the skin surface 20 is at least 1 mm or 1 surface data point per 1 mm², or the target resolution for the 6D-model of the skin surface is higher, e.g. at least 0.1 mm or 100 surface data points per 1 mm², respectively. In the case of a human face a surface area of approximately 20 cm×25 cm is captured, requiring at least 0.5 million or 5 million surface data points, respectively.

In step S4, the computer system 1 or its processor 100, respectively, generates a 6D-model of the skin surface 20. More specifically, the computer system 1 or its processor 100, respectively, generates the 6D-model of the skin surface 20 using the captured and stored 3D-surface images and their respective angular orientation (α1/β1, α2/β2) and distances (d1, d2). The 6D-model of the skin surface 20 is defined with respect to the global coordinate system, more specifically the reference coordinate system C (x, y, z). The 6D-model of the skin surface 20 comprises a plurality of surface data points. Each surface data point of the 6D-model comprises 3D-coordinates [x₁, y₁, z₁ . . . xₙ, yₙ, zₙ] and RGB-color information [r₁, g₁, b₁ . . . rₙ, gₙ, bₙ], as illustrated below in matrix K, representing the 6D-model of the skin surface 20:

$$K = [ \begin{matrix}{x}_{1} {y}_{1} {z}_{1} {r}_{1} {g}_{1} {b}_{1} \\ {x}_{2} {y}_{2} {z}_{2} {r}_{2} {g}_{2} {b}_{2} \\ … \\ {x}_{n} {y}_{n} {z}_{n} {r}_{n} {g}_{n} {b}_{n}\end{matrix} ]$$

To generate the 6D-model K of the skin surface 20, the computer system 1 or its processor 100, respectively, selects from the captured and stored 3D-surface images a 3D-reference image. The 3D-reference image has an angular orientation closest to the initial coordinate system C. In other words, the 3D-reference image is the 3D-surface image which was captured with the optical axis of the digital camera closest aligned to the z-axis of the initial coordinate system C, corresponding to the top view illustrated schematically in FIG. 8. The 3D-reference image is not being transformed, it is kept and remains unchanged. The computer system 1 or its processor 100, respectively, uses the selected 3D-reference image to generate the 6D-model K of the skin surface 20. More specifically, starting with the 3D-reference image, the computer system 1 or its processor 100, respectively, determines from the captured and stored 3D-surface images the adjacent 3D-surface images. Two adjacent (or neighboring) 3D-surface images have the closest angular orientation and distance with respect to each other. To generate the 6D-model K of the skin surface 20, the computer system 1 or its processor 100, respectively, rotates and translates (moves) adjacent 3D-reference images towards the 3D-reference image. Thus, starting with the 3D-surface image adjacent to the 3D-reference image, the adjacent 3D-surface image is rotated to align with the angular orientation of the 3D-reference image, and translated (moved) to the same distance as the 3D-reference image. To generate the 6D-model K of the skin surface 20, the computer system 1 or its processor 100, respectively, determines the rotational and/or translational transformation by optimizing the match of overlapping 3D data points (x, y, z) of the (ordered) skin surface features, also referred to as “3D landmark data”, of the two adjacent 3D-surface images being processed. For example, the transformation is optimized with regards to a best fit or least error measure. The computer system 1 or its processor 100, respectively, stores surface data points for the 6D-model K of the skin surface 20 when the match of the overlapping 3D data of the landmarks is within the optimization thresholds, e.g. within the best fit or error thresholds.

As illustrated in FIGS. 3, 4, 5 and 6, in step S5, the computer system 1 uses the 6D-model K of the skin surface 20 to generate a dermatological evaluation. If, after processing of all the captured and recorded 3D-surface images, the resulting 6D-model K of the skin surface 20 is insufficient with regards to desired quality, e.g. resolution or number of surface data points, the computer system 1 or its processor 100, respectively, will instruct the user to proceed in step S1* and S1 to capture and record further 3D-surface images, as indicated schematically by step S40 in FIGS. 3 and 4.

As is illustrated schematically in FIG. 3, steps S1, S1*, S2, S2*, S3, S4, S5, and S6 are executed by the computer system 1. In an embodiment, step S5 is executed by the electronic device 10 of the computer system 1 or its processor 100, respectively.

As is illustrated schematically in the embodiment or configuration of FIG. 4, steps S1, S1* S2, S2*, S3, S4, and S6 are executed by the electronic device 10 of the computer system 1, or its processor 100, respectively. In step S7, the 6D-model K of the skin surface 20 is transmitted from the electronic device 10 via communication network 2 to the computerized processing system 11. Step S5 is executed by the computerized processing system 11 of the computer system 1 or its processor 100*, respectively.

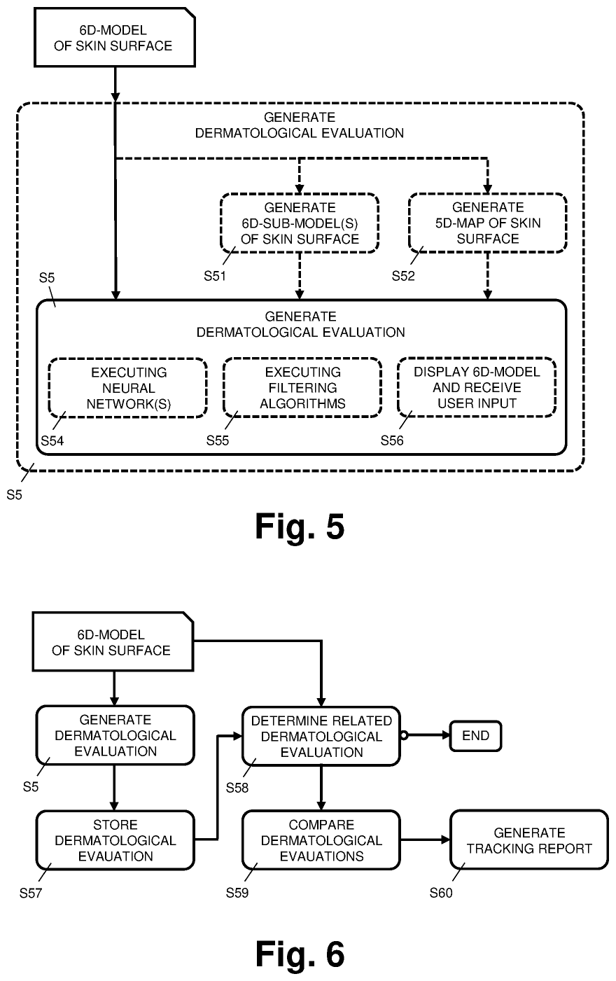

As illustrated in FIG. 5, in different embodiments, step S5 further comprises the computer system 1 executing optional step S51, for generating one or more 6D-sub-models of the skin surface 20, and/or optional step S52, for generating a 5D-map of the skin surface 20.

In step S51, the computer system 1 generates one or more 6D-sub-models of the skin sur-face 20. In essence, a 6D-sub-model of the skin surface 20 is an extracted area of the 6D-model of the skin surface 20. The 6D-sub-model or the extracted area, respectively, is defined by a central area point and an area radius, by a feature outline F1, F2, F3 of a specific skin surface, by a defined 2D area outline, and/or by defined 3D area outline, for example. A 6D-sub-model of the skin surface 20 comprises a plurality of surface data points of the respective area, and each surface data point comprises 3D-coordinates and RGB-color information. In the case of a human face, examples of 6D-sub-models or extracted areas include an eye, a nose, a mouth, a cheek, a forehead, or parts thereof, etc.

Each surface data point of the 6D-sub-model comprises 3D-coordinates [xᵤ, yᵤ, zᵤ . . . xᵥ, yᵥ, zᵥ] and RGB-color information [rᵤ, gᵤ, bᵤ . . . rᵥ, gᵥ, bᵥ], as illustrated below in matrix S, representing a 6D-sub-model of the skin surface 20:

$$S = [ \begin{matrix}{x}_{u} {y}_{u} {z}_{u} {r}_{u} {g}_{u} {b}_{u} \\ … \\ … \\ {x}_{v} {y}_{v} {z}_{v} {r}_{v} {g}_{v} {b}_{v}\end{matrix} ]$$

In step S52, the computer system 1 generates a 5D-map of the skin surface 20. More specifically, the computer system 1 generates the 5D-map of the skin surface 20 by applying a projection to the 6D-model of the skin surface 20. For example, the projection is performed along the z-axis of the reference coordinate system C or the reference image. Other projection axes are possible. The 5D-map of the skin surface 20 comprises a plurality of map data points. Each map data point comprises 2D-coordinates and RGB-color information.

Each map data point of the 5D-map comprises 2D-coordinates [xᵤ, yᵤ . . . xᵥ, yᵥ] and RGB-color information [rᵤ, gᵤ, bᵤ . . . rᵥ, gᵥ, bᵥ], as illustrated below in matrix M, representing a 5D-map of the skin surface 20:

$$M = [ \begin{matrix}{x}_{u} {y}_{u} {r}_{u} {g}_{u} {b}_{u} \\ … \\ … \\ {x}_{v} {y}_{v} {r}_{v} {g}_{v} {b}_{v}\end{matrix} ]$$

As illustrated schematically in FIG. 5, the computer system 1 generates the dermatological evaluation by executing a neural network in step S54, by executing various filtering algorithms in step S55, and/or by rendering the 6D-model of the skin surface 20 on a display and receiving evaluation data from a user via a user interface 103, 104.

The following table summarizes the various possible embodiments and/or configurations of input data K, S, and/or M and steps S54, S55, S56 for generating the dermatological evaluation:

| | | 6D-SUB- | |
|---|---|---|---|
| | 6D-MODEL | MODEL(S) | 5D-MAP |
| | OF SKIN | OF SKIN | OF SKIN |
| | SURFACE | SURFACE | SURFACE |
| | K | S | M |
| EXECUTE NEURAL | X | | |
| NETWORK S54 | | X | |
| and/or | | | X |
| EXECUTE FILTER | X | X | |
| ALGORITHM(S) S55 | X | | X |
| and/or | | X | X |
| DISPLAY 6D-MODEL | X | X | X |
| AND RECEIVE | | | |
| USER INPUT S56 | | | |

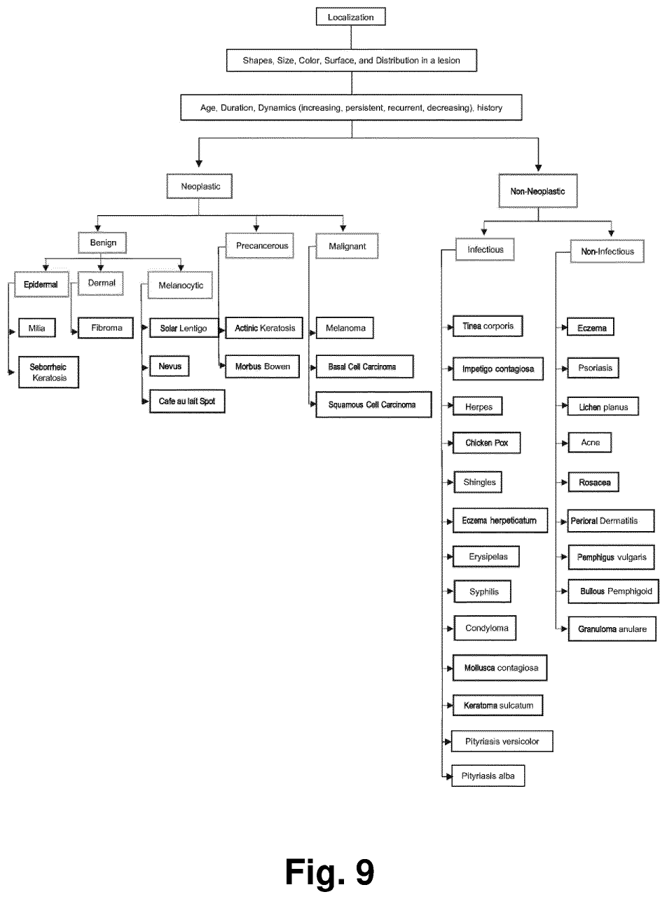

In step S54, the computer system 1 executes a neural network. For example, the neural network is a multi-label deep feed forward neural network, a convoluted neural network, or a transformer neural network. The neural network is trained to identify skin diseases, skin issues, and/or skin types, using a plurality of 6D-models K of skin surfaces 20 from a plurality of people, a plurality of 6D-sub-models S of skin surfaces 20 from a plurality of people, and/or a plurality of 5D-maps M of skin surfaces 20 from a plurality of people. Accordingly, the computer system 1 executes the neural network using the 6D-model K of the skin surface 20 generated in step S4, one or more 6D-sub-models S of the skin surface 20 generated in step S51, and/or the 5D-map M of the skin surface 20 generated in step S52. In an embodiment, the neural network is trained to and identifies the skin diseases, skin issues, and/or skin types, with a related probability. For example, the neural network uses a tree structure for labeling the skin diseases, skin issues, and/or skin types, as illustrated in FIG. 9. In a further embodiment, the neural network is trained and executed to detect skin surface characteristics, as described below in connection with step S55.

In step S55, the computer system 1 executes various filter algorithms to determine skin sur-face characteristics, particularly skin surface characteristics associated with specific skin diseases, skin issues, and/or skin types. The computer system 1 determines skin sur-face characteristics by applying the various filter algorithms to the 6D-model K of the skin surface 20 generated in step S4, to the one or more 6D-sub-models S of the skin surface 20 generated in step S51, and/or to the 5D-map M of the skin surface 20 generated in step S52. For example, the skin surface characteristics include wrinkles, wrinkle dimensions, pores, pore dimensions, milia, nodules, nodule dimensions, nodule shapes, macula, macula dimensions, macula shapes, papule, papule dimensions, papule shapes, pustules, pustule dimensions, blisters, blister dimensions, wheal, wheal dimensions, wheal shapes, comedo, erosions, erosion dimensions, erosion shapes, ulcers, ulcer dimensions, ulcer shapes, crust, scale, scale types, rhagade, and/or atrophy. The filter algorithms include algorithms for determining depth gradient (first derivative) on each surface data point of the 6D-model or 6D-sub-model, for determining a curvature measure (second derivative) of on each surface data point of the 6D-model or 6D-sub-model, for determining a color spectrum measure of regional data of the 6D-model or 6D-sub-model, for determining statistical measures of the gradient and curvature data such as mean and/or standard deviation, for determining density by measuring areas with changes in gradient and curvature, and/or for combined measures of color histograms with surface variation selection, etc.

For example, the computer system 1 applies linear type filters, such as gradient computation indicating the change in elevation from one pixel point to its geometric neighbors. The numerical differential is computed in all three dimensions by finite discretization. By computing a statistical measure, such as mean or variance, the average change or variance in change is determined, e.g. for specific region such as cheek or eye area. Different values for the average gradient and variance of the gradient determine the different skin sur-face characteristics. More complex filters are used for identifying more complex skin sur-face characteristics associated with specific diseases, such as squamous cell carcinomas. For example, these filters are configured to detect rings in the 5D-map of the skin surface 20 or dome-shape structures, associated with carcinomas, in the 6D-model or 6D-sub-models of the skin surface. For 2D detection, the filters include edge detection filters that detect significant changes of the gradient, i.e. above a gradient threshold value, in combination with structural filters that determine correlation with circular structures. The same approach is done in 3D by computing local gradients and detecting points with significant changes. For detecting skin surface characteristics with more complex geometric forms, the neural network(s) described above is trained and used accordingly.

In step S56, the computer system 1 renders the 6D-model of the skin surface 20 on a display. The rendering on the display is adjusted, for example, in response to user navigational commands, such as rotate left, rotate right, rotate up, rotate down, zoom in, zoom out, etc. Based on a visual analysis of the 6D-model of the skin surface 20, the computer system 1 obtains evaluation data from the user via a user interface 103, 104. In an embodiment, the user evaluation data is responsive and/or complementary to dermatological evaluation data generated by the computer system 1.

Further in step S5, the computer system 1 generates a dermatological evaluation of the 6D-model of the skin surface 20. More specifically, the computer system 1 generates the dermatological evaluation of the 6D-model of the skin surface 20, using the skin diseases, skin issues, and/or skin types, identified in step S54, the related probabilities determined in step S54, the skin surface characteristics determined in steps S54 and/or S55, and/or the user evaluation data received in step S56. The dermatological evaluation identifies skin diseases, skin issues, and/or skin types. In an embodiment, the dermatological evaluation identifies several skin diseases, skin issues, and/or skin types, with related probabilities. The computer system 1 stores the dermatological evaluation generated in step S5. In an embodiment, the dermatological evaluation further comprises an indication of remedies and/or skin care and/or treatment products related to the listed skin diseases, skin issues, and/or skin types, as retrieved from a database and included by the computer system 1.

In the embodiment or configuration of FIG. 6, responsive to obtaining or receiving a 6D-model of the skin surface 20 for evaluation, in step S58, the computer system 1 determines whether a related dermatological evaluation was stored previously for the particular individual. In an embodiment, the particular individual is identified by the computer system 1 using the obtained or received 6D-model of the skin surface 20 to be evaluated. More specifically, the computer system 1 compares the obtained or received 6D-model to previously evaluated and stored 6D-models of skin surfaces 20. A match of the obtained or received 6D-model with a previously evaluated and stored 6D-model is determined by the computer system 1 based on a similarity measure exceeding a defined similarity threshold value. The similarity measure is based on defined criteria, e.g. biometric characteristics determined from the obtained and stored 6D-models of the skin surface.

As illustrated in FIG. 6, in case the computer system 1 identifies a related dermatological evaluation which was previously generated and stored for the particular individual, processing continues in step S59.

In step S59, the computer system compares the current dermatological evaluation, which was generated in step S5 and stored in step S58, to the related dermatological evaluation, which was previously generated and stored for the particular individual.

In step S60, depending on the comparison of step S59, the computer system 1 generates a tracking report. The tracking report indicates changes that were identified by comparing the current dermatological evaluation and the related, previously generated dermatological evaluation for the particular individual. The changes included in the tracking report include positive changes, i.e. improvements of dermatological diseases and issues, and/or negative changes, i.e. deteriorations and/or new detections of dermatological diseases and issues. The computer system 1 includes the tracking report in the dermatological evaluation.

In step S6, the computer system 1 renders on a user interface 103 the dermatological evaluation generated in step S5. In an embodiment, step S6 is executed by the electronic device of the computer system 1 or its processor 100, respectively. As is illustrated schematically in the embodiment or configuration of FIG. 4, in step S8, the dermatological evaluation is transmitted from the computerized processing system 11 via communication network 2 to the electronic device 10.

It should be noted that the disclosed computer-implemented method of monitoring human skin and the related computer system 1 provide a measurement-based identification of skin diseases, skin issues, and/or skin types, as well as an assessment the skin type of a human. The disclosed method and computer system 1 make it possible to track the performance (over time) of skincare products, for medical and/or cosmetic purposes. The disclosed method and computer system 1 can also be applied in telemedicine for remote recording and remote viewing of the skin of a human, particularly the facial skin of a human, by a medical trained specialist. The 6D-model of skin surfaces 20 can be displayed photo realistically in 3D and provides the specialist a tool to assess the skin by zooming, rotating, and viewing the 6D-model of skin surfaces 20.

It should further be noted that, in the description, the sequence of the steps has been presented in a specific order, one skilled in the art will understand, however, that the order of at least some of the steps could be altered, without deviating from the scope of the disclosure.

1. A computer-implemented method of monitoring human skin, the method comprising:
Recording, by a camera and a depth sensor, a plurality of 3D-surface images of a skin surface, each 3D-surface image being taken from a different angle and a different distance with respect to the skin surface and comprising a plurality of pixels with depth information and RGB-color information;
detecting, by a computer system, skin surface features in the 3D-surface images;
determining, by the computer system, respective angular orientation and distance of the 3D-surface images, using the skin surface features and the depth information;
generating, by the computer system, a 6D-model of the skin surface, using the 3D-surface images and their respective angular orientation and distance, the 6D-model of the skin surface comprising a plurality of surface data points, each surface data point comprising 3D-coordinates and RGB-color information;
generating, by the computer system, a dermatological evaluation of the 6D-model of the skin surface; and
rendering, by the computer system, the dermatological evaluation on a user interface.

2. The method of claim 1, wherein detecting the skin surface features comprises the computer system determining from the 3D-surface images a body part, and using an initial coordinate system and respective feature outlines, associated with the body part, for detecting the skin surface features in the 3D-surface images.

3. The method of claim 2, further comprising the computer system selecting from the plurality of 3D-surface images a 3D-reference image, the 3D-reference image having a coordinate system with an angular orientation comparatively closest to the initial coordinate system, and generating the 6D-model of the skin surface using the 3D-reference image.

4. The method of claim 3, wherein generating the 6D-model of the skin surface comprises the computer system determining adjacent 3D-surface images, starting with the 3D-reference image, whereby two adjacent 3D-surface images have a comparatively closest angular orientation and distance to each other, and rotating and translating adjacent 3D-reference images towards the 3D-reference image, starting with the 3D-surface image adjacent to the 3D-reference image, to match their respective angular orientation and distance.

5. The method of claim 1, wherein generating the dermatological evaluation comprises the computer system including in the dermatological evaluation a probabilistic ranking of dermatological diagnoses related to skin diseases, skin issues, and/or skin types.

6. The method of claim 1, further comprising the computer system generating the dermatological evaluation using the 6D-model of the skin surface as input to a neural network, trained to identify skin diseases, skin issues, and/or skin types, using a plurality of 6D-models of skin surfaces from a plurality of people, which 6D-models of skin surfaces comprise a plurality of surface data points with 3D-coordinates and RGB-color information.

7. The method of claim 1, further comprising the computer system generating a 5D-map of the skin surface, by applying a projection to the 6D-model of the skin surface, the 5D-map of the skin surface comprising a plurality of map data points, each map data point comprising 2D-coordinates and RGB-color information; and generating the dermatological evaluation using the 5D-map of the skin surface as input to a neural network, trained to identify skin diseases, skin issues, and/or skin types, using a plurality of 5D-maps of skin surfaces from a plurality of people, which 5D-maps of skin surfaces comprise a plurality of map data points with 2D-coordinates and RGB-color information.

8. The method of claim 1, further comprising the computer system generating one or more 6D-sub-models of the skin surface, by extracting one or more areas of the 6D-model of the skin surface, each of the 6D-sub-models of the skin surface comprising a plurality of surface data points of the respective area, each surface data point comprising 3D-coordinates and RGB-color information; and generating the dermatological evaluation using the one or more 6D-sub-models of the skin surface as input to a neural network, trained to identify skin diseases, skin issues, and/or skin types, using a plurality of one or more of the 6D-sub-models of skin surfaces from a plurality of people, which one or more 6D-sub-models of skin surfaces comprise a plurality of surface data points of the respective area with 3D-coordinates and RGB-color information.

9. The method of claim 1, further comprising the computer system generating at least one of: one or more 6D-sub-models of the skin surface or a 5D-map of the skin surface, the one or more 6D-sub-models of the skin surface being generated by extracting one or more areas of the 6D-model of the skin surface, each of the 6D-sub-models of the skin surface (20) comprising a plurality of surface data points of the respective area, each surface data point comprising 3D-coordinates and RGB-color information, and the 5D-map of the skin surface being generated by applying a projection to the 6D-model of the skin surface, the 5D-map of the skin surface comprising a plurality of map data points, each map data point comprising 2D-coordinates and RGB-color information; and generating the dermatological evaluation using at least one of: the 6D-model of the skin surface, the one or more 6D-sub-models of the skin surface, or the 5D-map of the skin surface, as input to a neural network, trained to identify skin diseases, skin issues, and/or skin types, using at least one of: a plurality of 6D-models of skin surfaces from a plurality of people, a plurality of one or more 6D-sub-models of skin surfaces from a plurality of people, or a plurality of 5D-maps of skin surfaces from a plurality of people.

10. The method of claim 1, further comprising the computer system using the 6D-model of the skin surface to determine skin surface characteristics, the skin surface characteristics including at least one of: wrinkles, wrinkle dimensions, pores, pore dimensions, milia, nodules, nodules, nodule dimensions, nodule shapes, macula, macula dimensions, macula shapes, papule, papule dimensions, papule shapes, plaque, plaque dimensions, plaque shape, pustules, pustule dimensions, blisters, blister dimensions, wheal, wheal dimensions, wheal shapes, comedo, erosions, erosion dimensions, erosion shapes, ulcers, ulcer dimensions, ulcer shapes, crust, scale, scale types, rhagade, or atrophy; and to generate the dermatological evaluation using the skin surface characteristics.

11. The method of claim 1, further comprising the computer system, upon completion of a first dermatological evaluation for a first 6D-model of the skin surface, storing in a data storage the first 6D-model of the skin surface and the first dermatological evaluation; upon receiving a second 6D-model of the skin surface, identifying in the data storage the first dermatological evaluation, by comparing the second 6D-model of the skin surface to the first 6D-model of the skin surface; upon completion of a second dermatological evaluation for the second 6D-model of the skin surface, generating a tracking report by comparing the second dermatological evaluation to the first dermatological evaluation; and rendering the tracking report on the user interface.

12. The method of claim 1, wherein generating the dermatological evaluation comprises the computer system rendering the 6D-model of the skin surface on a display, receiving evaluation data from a user via a user interface, and generating the dermatological evaluation using the evaluation data.

13. The method of claim 1, wherein generating the dermatological evaluation comprises an electronic device of the computer system transmitting the 6D-model of the skin surface via communication network to a processing system of the computer system, the processing system generating the dermatological evaluation using the 6D-model of the skin surface received from the electronic device, and the processing system transmitting the dermatological evaluation to the electronic device.

14. A computer system comprising a camera, a depth sensor, a user interface, and one or more processors, the one or more processors being configured to perform the following steps:
controlling the camera and the depth sensor to record a plurality of 3D-surface images of a skin surface, each 3D-surface image being taken from a different angle and a different distance with respect to the skin surface and comprising a plurality of pixels with depth information and RGB-color information;
detecting skin surface features in the 3D-surface images;
determining respective angular orientation and distance of the 3D-surface images, using the skin surface features and the depth information;
generating a 6D-model of the skin surface, using the 3D-surface images and their respective angular orientation and distance, the 6D-model comprising a plurality of surface data points, each surface data point comprising 3D-coordinates and RGB-color information;
generating a dermatological evaluation of the 6D-model of the skin surface; and
rendering the dermatological evaluation on the user interface.

15. The computer system of claim 14, wherein the one or more processors are configured to perform a method according to one of the claims 1 to 12.

16. The computer system of claim 14, comprising an electronic device and a processing system, the electronic device including at least one of the one or more processors configured to transmit the 6D-model of the skin surface via communication network to the processing system, and the processing system, including at least another one of the one or more processors configured to generate the dermatological evaluation, using the 6D-model of the skin surface received from the electronic device, and to transmit the dermatological evaluation to the electronic device.

17. A computer program product comprising computer program code configured to control a processor of an electronic device comprising a camera, a depth sensor, and a user interface connected to the processor, such that the processor performs the following steps:
control the camera and the depth sensor to record a plurality of 3D-surface images of a skin surface of a human skin, each 3D-surface image being taken from a different angle and a different distance with respect to the skin surface and comprising a plurality of pixels with depth information and RGB-color information;
detect skin surface features in the 3D-surface images;
determine respective angular orientation and distance of the 3D-surface images, using the skin surface features and the depth information;
generate a 6D-model of the skin surface, using the 3D-surface images and their respective angular orientation and distance, the 6D-model of the skin surface comprising a plurality of surface data points, each surface data point comprising 3D-coordinates and RGB-color information;
transmit the 6D-model of the skin surface to a computerized processing system;
receive from the computerized processing system a dermatological evaluation of the 6D-model of the skin surface; and
render the dermatological evaluation on the user interface.

18. The computer program product of claim 17, wherein the computer program code is further configured to control the processor to perform a method according to one of the claims 1 to 12.

19. A computer program product comprising computer program code configured to control one or more processors of a computerized processing system to perform the following steps:
receiving from an electronic device a 6D-model of a skin surface of a person, the 6D-model of the skin surface comprising a plurality of surface data points, each surface data point comprising 3D-coordinates and RGB-color information;
generating a dermatological evaluation of the 6D-model of the skin surface of the person; and
transmitting the dermatological evaluation to the electronic device.

20. The computer program product of claim 19, wherein the computer program code is further configured to control the one or more processors to perform a method according to one of the claims 5 to 12.
